(19) United States
(12) Patent Application Publication
(10) Pub. No.: US 2020/0305846 A1
(43) Pub. Date: Oct. 1, 2020

(54) METHOD AND SYSTEM FOR RECONSTRUCTING TRACHEA MODEL USING ULTRASONIC AND DEEP-LEARNING TECHNIQUES

(71) Applicant: Fei-Kai Syu, Pingtung City (TW)

(72) Inventor: Fei-Kai Syu, Pingtung City (TW)

(21) Appl. No.: 16/367,283

(22) Filed: Mar. 28, 2019

(51) Int. Cl. A61B 8/08; A61B 8/12; G06T 7/00; G06T 17/00

(52) U.S. Cl. CPC A61B 8/5207; A61B 8/12; G06T 7/0012; G06T 2210/41; G06T 2207/10012; G06T 2207/10136; G06T 2207/30061; G06T 17/00

(57) ABSTRACT

A tracheal model reconstruction method using the ultrasonic and deep-learning techniques; which comprises the following steps: obtaining the image and position information of the tracheal wall, positioning the graph-information space, processing image, extracting the image-feature and recognizing the image using deep-learning, positioning the 6 DoF space, calibrating the image-space, converting the image-space, and forming a three-dimensional trachea model. Thereby, providing a tracheal model reconstruction method that can correctly and quickly reconstruct and record a stereoscopic three-dimensional tracheal model.

### (a) TECHNICAL FIELD OF THE INVENTION

The present invention is a tracheal model reconstruction method and system thereof using the ultrasonic and deep-learning techniques, and especially relates to a tracheal model reconstruction method and system thereof capable of correctly and quickly reconstructing and recording a stereoscopic three-dimensional trachea model.

### (B) DESCRIPTION OF THE PRIOR ART

When the patient underwent the general anesthesia, the cardiopulmonary resuscitation, or the patient is unable to breathe on their own during surgery, the patient must be intubated to insert the artificial airway into the trachea; so that the medical gas is smoothly delivered into the patient's trachea.

When the patient underwent the general anesthesia, the cardiopulmonary resuscitation, or the patient is unable to breathe on their own during surgery, the patient must be intubated to insert the artificial airway into the trachea; so that the medical gas is smoothly delivered into the patient's trachea.

Therefore, the rapid and correct establishment of a three-dimensional trachea model for providing the medical personnel to assist intubation is an urgent problem to be solved.

### SUMMARY OF THE INVENTION

The object of the present invention is to improve the above-mentioned defects, and to provide a tracheal model reconstruction method and system thereof capable of correctly and quickly reconstructing and recording a stereoscopic three-dimensional trachea model.

In order to achieve the above object, the tracheal model reconstruction method using the ultrasonic and deep-learning techniques of the present invention comprises the following steps:

obtaining the image and position information of the tracheal wall: An ultrasonic image is obtained by scanning the oral cavity to the trachea by using a positionable ultrasonic scanner, and the position information of the ultrasonic image is synchronously obtained according to the scanning position;

positioning the graph-information space: the spatial positioning processing of the ultrasonic image is performed, and the spatial positioning information of the ultrasonic image is obtained;

processing image: the ultrasonic image is denoised, noise-decreased, and cropped; and the image is proceeded enhancement to enhance the details of the ultrasonic image for obtaining a clear ultrasonic image;

extracting the image-feature and recognizing the image using deep-learning:

extract and capture the clear ultrasonic image, and store a variety of different image features and a continuous tracheal wall image; and then through training the deep-learning model to achieve assisting the identification of the image features and the tracheal wall image; and position the shape, curvature, and position of the tracheal wall;

positioning the 6 DoF space: the ultrasonic image and the spatial position information in the step of positioning the graph-information space are proceeded the positioning process to obtain the spatial positioning data of the ultrasonic image;

calibrating the image-space: calibrate the positioning data of the ultrasonic image-space after the positioning process of the step of positioning the 6 DoF space to obtain the actual size and actual projection position of the ultrasonic image in the three-dimensional space to convert to the actual three-dimensional spatial position, and calibrate the correct size of the output ultrasonic image;

converting the image-space: the ultrasonic image in the step of calibrating the image-space is projected into the three-dimensional space to obtain the three-dimensional spatial data and the image information of the trachea model; and

forming a three-dimensional trachea model: the ultrasonic image obtained in the step of extracting the image-feature and recognizing the image using deep-learning is connected with the three-dimensional spatial data and the image information of the trachea model obtained in the step of converting the image-space; and they are spliced, reconstructed, and recorded to form an actual stereoscopic three-dimensional trachea model.

Thereby, providing a tracheal model reconstruction method that can correctly and quickly reconstruct and record a stereoscopic three-dimensional tracheal model for subsequent medical research or use.

### BRIEF DESCRIPTION OF THE DRAWINGS

FIG. 1 is a step flow chart of the present invention.

FIG. 2 is a system block diagram of the present invention.

FIG. 3 is a system block diagram of the present invention combined with a positionable ultrasound scanner.

### DETAILED DESCRIPTION OF THE PREFERRED EMBODIMENTS

The following descriptions are exemplary embodiments only, and are not intended to limit the scope, applicability or configuration of the invention in any way. Rather, the following detailed description provides a convenient illustration for implementing exemplary embodiments of the invention. Various changes to the described embodiments may be made in the function and arrangement of the elements described without departing from the scope of the invention as set forth in the appended claims.

The foregoing and other aspects, features, and utilities of the present invention will be best understood from the following detailed description of the preferred embodiments when read in conjunction with the accompanying drawings.

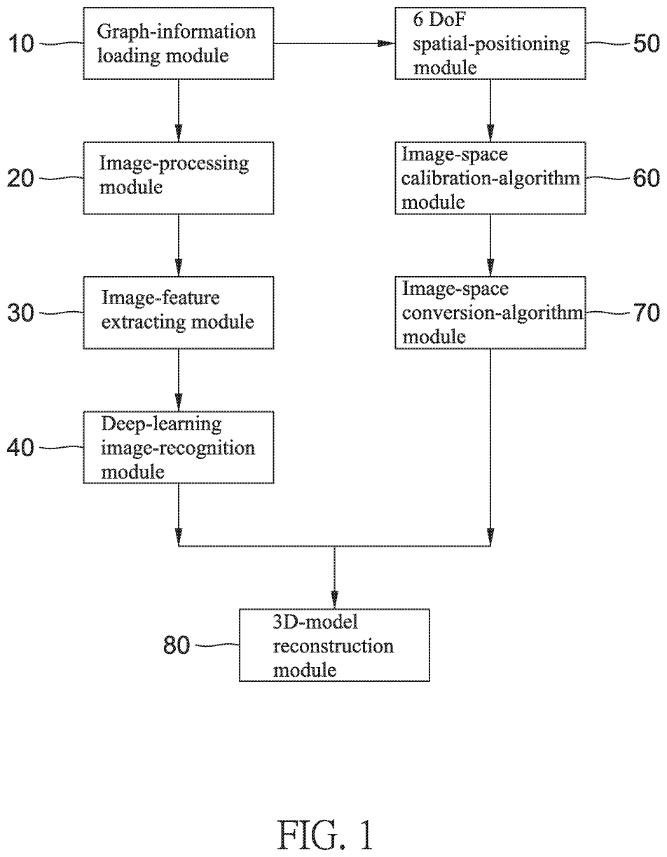

Regarding the technical means and the structure applied by the present invention to achieve the object, the embodiment shown in FIG. 1 to FIG. 3 will be explained in detail as follows, as shown in the step flow chart shown in FIG. 1, the steps are explained in detail as follows.

Obtaining the image and position information of the tracheal wall: An ultrasonic image is obtained by scanning the oral cavity to the trachea by using a positionable ultrasonic scanner, and the position information of the ultrasonic image is synchronously obtained according to the scanning position.

Positioning the graph-information space: The spatial positioning processing of the ultrasonic image is performed, and the spatial positioning information of the ultrasonic image is obtained.

Processing image: The ultrasonic image is denoised, noise-decreased, and cropped; and the image is proceeded enhancement to enhance the details of the ultrasonic image for obtaining a clear ultrasonic image.

Extracting the image-feature and recognizing the image using deep-learning: Extract and capture the clear ultrasonic image, and store a variety of different image features and a continuous tracheal wall image; and then through training the deep-learning model to achieve assisting the identification of the image features and the tracheal wall image; and position the shape, curvature, and position of the tracheal wall.

Positioning the 6 DoF space: The ultrasonic image and the spatial position information in the step of positioning the graph-information space are proceeded the positioning process to obtain the spatial positioning data of the ultrasonic image.

Calibrating the image-space: Calibrate the positioning data of the ultrasonic image-space after the positioning process of the step of positioning the 6 DoF space to obtain the actual size and actual projection position of the ultrasonic image in the three-dimensional space to convert to the actual three-dimensional spatial position, and calibrate the correct size of the output ultrasonic image.

Converting the image-space: The ultrasonic image in the step of calibrating the image-space is projected into the three-dimensional space to obtain the three-dimensional spatial data and the image information of the trachea model.

Forming a three-dimensional trachea model: The ultrasonic image obtained in the step of extracting the image-feature and recognizing the image using deep-learning is connected with the three-dimensional spatial data and the image information of the trachea model obtained in the step of converting the image-space; and they are spliced, reconstructed, and recorded to form an actual stereoscopic three-dimensional trachea model.

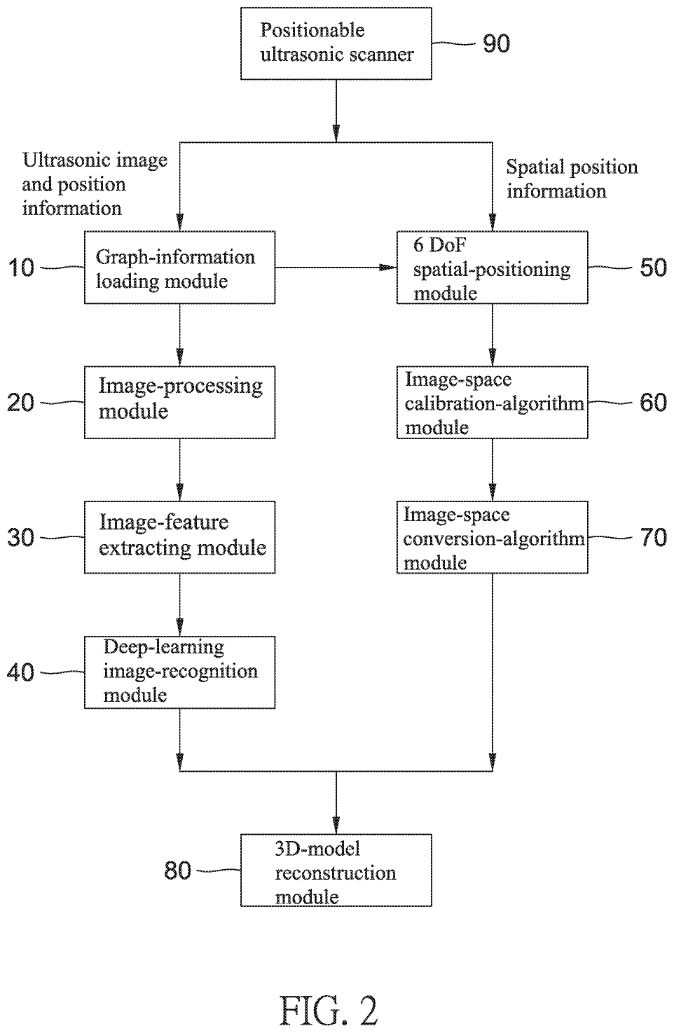

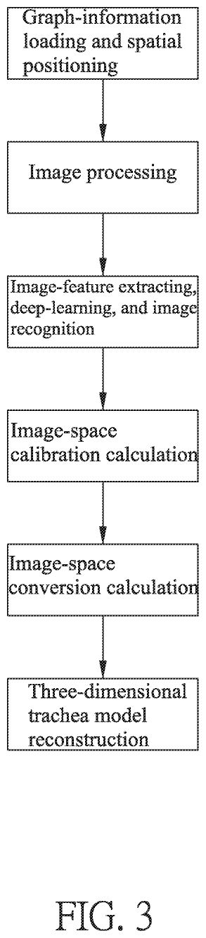

In order to further illustrate the present invention, the system configuration diagrams shown in FIG. 2 and FIG. 3 are further described in detail as follows.

As shown in FIG. 2, the tracheal model reconstruction system using the ultrasonic and deep-learning techniques of the present invention comprises a graph-information loading module 10, an image-processing module 20, an image-feature extracting module 30, a deep-learning image-recognition module 40, a 6 DoF spatial-positioning module 50, an image-space calibration-algorithm module 60, an image-space conversion-algorithm module 70, and a 3D-model reconstruction module 80; which are further described in detail as follows.

The graph-information loading module 10 (please simultaneously refer to FIG. 2) is connected with the positionable ultrasonic scanner 90 for loading the ultrasonic image and position information obtained by the positionable ultrasonic scanner 90, and collaborating with the space positioning to process image.

The image-processing module 20 (please simultaneously refer to FIG. 2) is connected with the graph-information loading module 10 for the ultrasonic image to be denoised, noise-decreased, and cropped; and the image is proceeded enhancement to enhance the details of the ultrasonic image for obtaining a clear ultrasonic image.

The image-feature extracting module 30 (please simultaneously refer to FIG. 2) is connected with the image-processing module 20 for capturing, extracting, and storing a variety of different image features of the clear ultrasonic image and a continuous tracheal wall image.

The deep-learning image-recognition module 40 (please simultaneously refer to FIG. 2) is connected with the image-feature extracting module 30; which is according to the variety of different image features and the continuous tracheal wall image stored in the image-feature extracting module 30, and is for training the deep-learning model to achieve assisting the identification of the tracheal wall in the ultrasonic image and achieve positioning the shape, curvature, and position information of partial tracheal wall of the planar clear ultrasonic image.

Continuing the above explanation; in a preferred embodiment, the deep-learning image-recognition module 40 can be designed to manually, automatically or semi-automatically control for positioning the shape, curvature, and position information of the tracheal wall.

The 6 DoF spatial-positioning module 50 (please simultaneously refer to FIG. 2) is connected with the graph-information loading module 10 and the positionable ultrasonic scanner 90 for receiving and loading the spatial position information obtained by the positionable ultrasonic scanner 90; and for performing the spatial positioning processing of the ultrasonic image loaded by the graph-information loading module 10 to obtain the ultrasonic image data and the spatial positioning information data.

The image-space calibration-algorithm module 60 (please simultaneously refer to FIG. 2) is connected with the 6 DoF spatial-positioning module 50; which is for receiving and calibrating the actual size and actual projection position in the three-dimensional space of the spatial positioning data processed by the 6 DoF spatial-positioning module 50 to convert into the actual three-dimensional spatial position and calibrate the correct size of the output ultrasonic image.

The image-space conversion-algorithm module 70 (please simultaneously refer to FIG. 2) is connected with the image-space calibration-algorithm module 60 for receiving the ultrasonic image processed by the image-space calibration-algorithm module 60; and for projecting the ultrasonic image into the three-dimensional space to obtain the three-dimensional spatial data and the image information of the trachea model.

The 3D-model reconstruction module 80 (please simultaneously refer to FIG. 2) is connected with the deep-learning image-recognition module 40 and the image-space conversion-algorithm module 70 for receiving the clear ultrasonic image of the deep-learning image-recognition module 40; and which is according to the three-dimensional spatial data and image information of the trachea model obtained by the image-space conversion-algorithm module 70 to make the clear ultrasonic image of the continuous tracheal wall be connected and spliced, so that the complete stereoscopic three-dimensional trachea model is reconstructed and recorded.

In addition, the image-feature extracting and deep-learning image-recognition steps and the deep-learning image-recognition module 40 use a plurality of patients' tracheal image data to extract and capture the image features; and input the image features and ultrasonic images into the deep-learning model; which The deep-learning model can be selected from the group consisting of supervised learning, unsupervised learning, semi-supervised learning, and reinforced learning (e.g., neural networks, random forest, support vector machine SVM, decision tree, or cluster, etc.); so that it can recognize the features, shape, curvature, and position of the tracheal wall through the deep-learning model.

Thereby, the present invention uses a positionable ultrasonic scanner to obtain continuous ultrasonic image and corresponding position information, and then forms clear ultrasonic image and image features through the image processing and feature extraction; and collaborating with the deep-learning to recognize the shape, curvature, and position of the tracheal wall; and using the 6 DoF spatial positioning and the image-space calibration and conversion to obtain the corresponding spatial information of the ultrasonic image; so that the stereoscopic three-dimensional trachea model can be correctly and quickly reconstructed and formed to provide for the intubation assistance and subsequent medical research or use.

I claim:

1. A tracheal model reconstruction method using the ultrasonic and deep-learning techniques, which comprises the following steps:
obtaining the image and position information of the tracheal wall: An ultrasonic image is obtained by scanning the oral cavity to the trachea by using a positionable ultrasonic scanner, and the position information of the ultrasonic image is synchronously obtained according to the scanning position;
positioning the graph-information space: the spatial positioning processing of the ultrasonic image is performed, and the spatial positioning information of the ultrasonic image is obtained;
extracting the image-feature and recognizing the image using deep-learning: extract and capture the clear ultrasonic image, and store a variety of different image features and a continuous tracheal wall image; and then through training the deep-learning model to achieve assisting the identification of the image features and the tracheal wall image; and position the shape and position of the tracheal wall;
positioning the 6 DoF space: the ultrasonic image and the spatial position information in the step of positioning the graph-information space are proceeded the positioning process to obtain the spatial positioning data of the ultrasonic image;
calibrating the image-space: calibrate the positioning data of the ultrasonic image-space after the positioning process of the step of positioning the 6 DoF space to obtain the actual size and actual projection position of the ultrasonic image in the three-dimensional space to convert to the actual three-dimensional spatial position, and calibrate the correct size of the output ultrasonic image;
converting the image-space: the ultrasonic image in the step of calibrating the image-space is projected into the three-dimensional space to obtain the three-dimensional spatial data and the image information of the trachea model; and
forming a three-dimensional trachea model: the ultrasonic image obtained in the step of extracting the image-feature and recognizing the image using deep-learning is connected with the three-dimensional spatial data and the image information of the trachea model obtained in the step of converting the image-space; and they are spliced, reconstructed, and recorded to form an actual stereoscopic three-dimensional trachea model.

2. A tracheal model reconstruction system using the ultrasonic and deep-learning techniques, which is applied to the tracheal model reconstruction method using the ultrasonic and deep-learning techniques of claim 1 and comprises a graph-information loading module, an image-processing module, an image-feature extracting module, a deep-learning image-recognition module, a 6 DoF spatial-positioning module, an image-space calibration-algorithm module, an image-space conversion-algorithm module, and a 3D-model reconstruction module; wherein:
the graph-information loading module is connected with the positionable ultrasonic scanner for loading the ultrasonic image and position information obtained by the positionable ultrasonic scanner, and collaborating with the space positioning to process image;
the image-feature extracting module is connected with the image-processing module for capturing, extracting, and storing a variety of image features of the clear ultrasonic image and a continuous tracheal wall image;
the deep-learning image-recognition module is connected with the image-feature extracting module; which is according to the image features and the continuous tracheal wall image stored in the image-feature extracting module, and is for training the deep-learning model to achieve assisting the identification of the tracheal wall in the ultrasonic image and achieve positioning the shape and position information of partial tracheal wall of the planar clear ultrasonic image;
the 6 DoF spatial-positioning module is connected with the graph-information loading module and the positionable ultrasonic scanner for receiving and loading the spatial position information obtained by the positionable ultrasonic scanner, and for performing the spatial positioning processing of the ultrasonic image loaded by the graph-information loading module to obtain the ultrasonic image data and the spatial positioning information data;
the image-space calibration-algorithm module is connected with the 6 DoF spatial-positioning module; which is for receiving and calibrating the actual size and actual projection position in the three-dimensional space of the spatial positioning data processed by the 6 DoF spatial-positioning module to convert into the actual three-dimensional spatial position and calibrate the correct size of the output ultrasonic image;
the image-space conversion-algorithm module is connected with the image-space calibration-algorithm module for receiving the ultrasonic image processed by the image-space calibration-algorithm module; and for projecting the ultrasonic image into the three-dimensional space to obtain the three-dimensional spatial data and the image information of the trachea model; and
the 3D-model reconstruction module is connected with the deep-learning image-recognition module and the image-space conversion-algorithm module for receiving the clear ultrasonic image of the deep-learning image-recognition module; and which is according to the three-dimensional spatial data and image information of the trachea model obtained by the image-space conversion-algorithm module to make the clear ultrasonic image of the continuous tracheal wall be connected and spliced, so that the complete stereoscopic three-dimensional trachea model is reconstructed and recorded.
